(19) United States
(12) Patent Application Publication
(10) Pub. No.: US 2004/0210202 A1
(43) Pub. Date: Oct. 21, 2004

(54) Aortic cannula

(76) Inventor: Gerald S. Weinstein, Las Cruces, NM (US)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/825,463

(22) Filed: Apr. 15, 2004

Related U.S. Application Data
(60) Provisional application No. 60/463,717, filed on Apr. 17, 2003.

(51) Int. Cl. A61M 25/00

(52) U.S. Cl. 604/264

(57) ABSTRACT

An aortic cannula has an elongate generally tubular side wall defining a conduit with distal and proximal ends. The distal end is adapted for insertion into the aorta during heart surgery to provide blood to the aorta. The conduit is separated into plural elongate lumens having smooth uninterrupted side walls and substantially equal cross sections extending back from the distal end of the cannula. The distal end is configured to permit substantially unrestricted laminar flow from all lumens out the distal end of the cannula.

### CROSS REFERENCE TO RELATED APPLICATION

This application claims priority to U.S. Provisional Patent Application No. 60/463,717, filed Apr. 17, 2003 and incorporates that application by reference.

### BACKGROUND OF THE INVENTION

During heart bypass surgery, it is necessary to direct oxygenated blood from the heart-lung machine into the aorta through an aortic cannula. Aortic cannulas are generally tubular structures with a diameter of about 7 to 8 mm which must deliver blood at the rate of about 5 to 6 liters per minute. It is important that the blood emerging into the aorta not scour the walls of the aorta dislodging material from the walls of the aorta causing embolisms. Moreover, the blood itself is fragile and may be damaged by stress caused by turbulence.

Any number of designs for aortic cannulas have been patented. However, little attention has been given to protection of the blood and insuring that the flow of the blood into the aorta is at as low a velocity as consistent with the volume flow rate and the diameter of the cannula and as laminar as possible while in the cannula and when exiting from the cannula. Current cannula designs reduce flow velocity but at the cost of increased turbulence.

It is an object, according to this invention, to provide an improved aortic cannula that provides low velocity laminar flow as the blood emerges into the aorta.

### SUMMARY OF THE INVENTION

Briefly, according to this invention, there is provided an aortic cannula having an elongate, generally tubular side wall defining a conduit with distal and proximal ends, the distal end being adapted for insertion into the aorta during heart surgery to provide blood to the aorta. The conduit is separated into plural elongate lumens having smooth uninterrupted side walls and substantially equal cross sections extending back from the distal end of the cannula such that the velocity of the blood flowing along the lumen will not change. It is further preferred that all lumens have substantially the same cross section so that the velocity of flow in each lumen is substantially identical. The interior walls of each lumen are curved to avoid sharp corners to avoid turbulence. The distal end is configured to permit substantially unrestricted axial flow out the distal end of the cannula from all lumens.

According to a preferred embodiment, at least three lumens spiral around each other near the distal end.

According to yet another embodiment of this invention, there is provided an aortic cannula having an elongate generally tubular side wall having distal and proximal ends. The distal end is adapted for insertion in the aorta. The distal end supports a rounded rigid web across the opening in the end of the cannula which serves as a deflector when the distal end is being inserted. The distal end is configured to permit substantially unrestricted laminar flow. Preferably, at least a portion of the opening at the distal end faces radially outward of the tubular side wall.

### BRIEF DESCRIPTION OF THE DRAWINGS

Further features and other objects and advantages will become clear from the following detailed description made with reference to the drawing in which:

FIG. 1 is a perspective view of an aortic cannula according to this invention;

FIG. 2 is a broken away side view of the aortic cannula shown in FIG. 1;

FIG. 3 is an end view of the aortic cannula shown in FIG. 1;

FIG. 4 is a section view taken along line IV-IV of FIG. 2; and

FIG. 5 is a broken away side view of an alternate embodiment.

### DESCRIPTION OF THE PREFERRED EMBODIMENTS

Referring to the drawings, there is shown the distal end of an aortic cannula 10 according to this invention. The cannula has a generally tubular side wall 11 which may be tens of centimeters long. The outside diameter of the side wall is about 8 to 10 millimeters. The distal end of the cannula narrows somewhat to form a tip 12 that has a rounded end 13 for enabling easy penetration into an opening cut in the wall of the aorta. The interior of the cannula is divided into a plurality of side-by-side lumens which extend back from the distal end. As shown in the drawing, there are three lumens 14a. 14b, and 14c. It is acceptable if the plural lumens open into a single lumen between the distal and the proximal end of the cannula. Each of the plural lumens has a substantially identical cross section and smooth uninterrupted walls that have no sharp changes in cross section.

It is preferred that the plural lumens have a substantially identical cross section end to end even as the cannula narrows at the distal end. This can be achieved by thinning the side wall and the walls separating the plural lumens at the distal end. The openings in the distal end out of the plural lumens must be at least as large as the cross section of the lumens. This enables the continued laminar flow out of the end of the cannula.

According to a preferred embodiment, the plural lumens spiral around each other especially near the distal end of the cannula. The spiraling of the lumens is the result of the helical shape of the walls separating the lumens within the cannula. Most preferably, the plural lumens spiral through at least 120 degrees. It is believed this will provide a slight spiraling motion to the blood emerging from the cannula which will assist in maintaining the laminar flow as the blood enters the aorta. In an alternate embodiment as shown in FIG. 5, the plural lumens do not spiral.

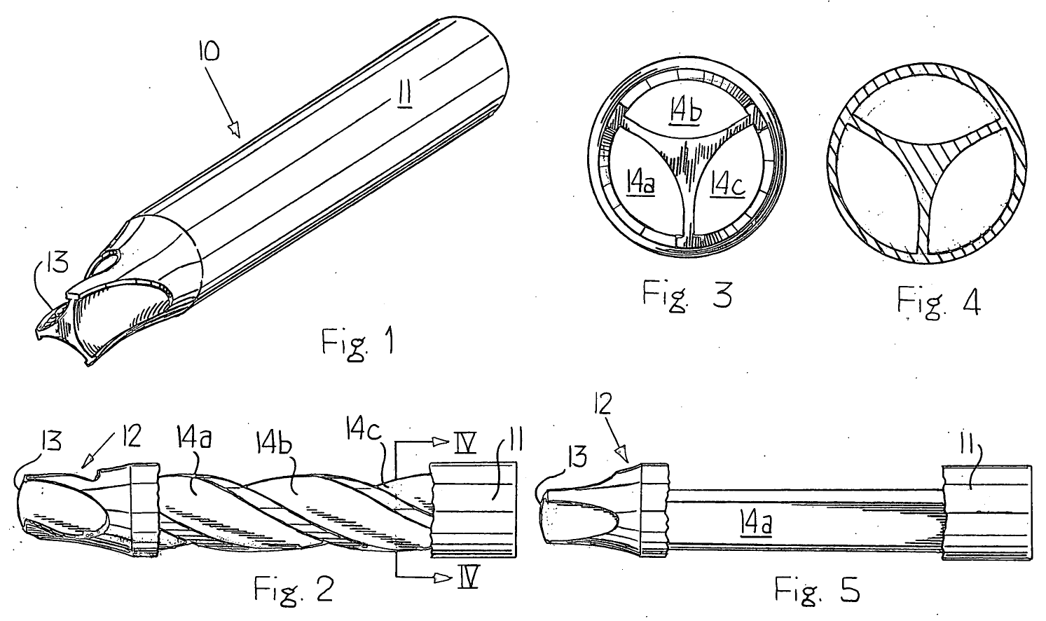

The walls separating the conduit into plural elongate lumens at the distal end form a rigid rounded web which serves as a deflector when the cannula is being inserted. A portion of the openings at the distal end face radially outward of the tubular side wall. As shown in FIG. 1, the tubular side wall gradually narrows near the distal end and the area of the openings at the distal end is at least as great as the cross-sectional area of the interior of the tubular side wall prior to where it narrows. FIG. 5 illustrates an embodiment wherein the conduit is divided into lumens that do not rotate. The walls still form a rigid rounded web which serves as a deflector. Indeed, it would be advantageous in an aortic cannula which is not divided into plural lumens to provide the rigid round web at only the very distal end of the cannula.

The aortic cannula can be fabricated by the synthetic materials that are commonly used in the manufacture of sterile cannulas and catheters. Typically, the cannula would be formed by extrusion.

Having thus described my invention with the detail and particularity required by the Patent Laws, what is desired protected by Letters Patent is set forth in the following claims.

The invention claimed is:

1. An aortic cannula having an elongate generally tubular side wall defining a conduit with distal and proximal ends, the distal end being adapted for insertion into the aorta during heart surgery to provide blood to the aorta, said conduit being separated into plural elongate lumens having smooth uninterrupted side walls and substantially equal cross sections extending back from the distal end of the cannula, said distal end being configured to permit substantially unrestricted laminar flow from all lumens out the distal end of the cannula.

2. The aortic cannula according to claim 1, having at least three lumens that spiral around each other near the distal end.

3. The aortic cannula according to claim 2, wherein the lumens spiral through at least 120 degrees.

4. The aortic cannula according to claim 1, wherein there are at least three lumens.

5. The aortic cannula according to any one of claims 1, 2, 3, or 4, which has a narrowed and rounded distal end.

6. The aortic cannula according to and one of claims 1, 2, 3, or 4 wherein the cross sections of the lumens are substantially identical end-to-end and to each other.

7. The aortic cannula according to claim 1, wherein walls separating the conduit into plural elongate lumens define a rounded web across the distal end which serves as a deflector when the distal end is being inserted in the aorta.

8. The aortic cannula according to claim 7, wherein at least a portion of the openings at the distal end face radially outward of the tubular side wall.

9. An aortic cannula according to claim 8, wherein the tubular side wall gradually narrows near the distal end and the area of the openings at the distal end is at least a great as the cross-sectional area of the interior of the tubular side wall prior to where it narrows.

10. An aortic cannula having an elongate generally tubular side wall defining a conduit with distal and proximal ends, the distal end being adapted for insertion in the aorta during heart surgery to provide blood to the aorta, the distal end supporting a rounded rigid web across the opening in the end thereof which serves as a deflector when the distal end is being inserted in the aorta and said distal end being configured to permit substantially unrestricted laminar flow.

11. The aortic cannula according to claim 10, wherein at least a portion of the opening at the distal end faces radially outward of the tubular side wall.

12. An aortic cannula according to claim 11, wherein the tubular side wall gradually narrows near the distal end and the area of the openings of the distal end is at least as great as the cross-sectional area of the interior of the tubular side wall prior to where it narrows.
